(19) United States
(12) Patent Application Publication
(10) Pub. No.: US 2002/0169183 A1
(43) Pub. Date: Nov. 14, 2002

(54) Acridines as stimulators for Fas-mediated apoptosis

(76) Inventors: Hugo O. Villar, La Jolla, CA (US); Edgardo Laborde, Foster City, CA (US)

Correspondence Address: HELLER EHRMAN WHITE & MCAULIFFE LLP, 275 MIDDLEFIELD ROAD, MENLO PARK, CA, 94025-3506, US

(21) Appl. No.: 10/082,801

(22) Filed: Feb. 22, 2002

Related U.S. Application Data
(60) Provisional application No. 60/274,535, filed on Mar. 8, 2001.

(51) Int. Cl. A61K 31/473

(52) U.S. Cl. 514/297

(57) ABSTRACT

Optionally substituted 9-[(3-aminopropyl)amino]acridines stimulate Fas-mediated apoptosis. These compounds, as single stereoisomers or mixtures of stereoisomers, their pharmaceutically acceptable salts, and pharmaceutical compositions containing them, are useful in methods of treating autoimmune and hyperplasic diseases.

### CROSS-REFERENCE TO RELATED APPLICATION

This application claims the priority under 35 USC 119(e) of U.S. Provisional Application No. 60/274,535, filed Mar. 8, 2001, the disclosure of which is incorporated into this application by reference.

### BACKGROUND OF THE INVENTION

1. Field of the Invention

The present invention relates to the use of certain acridines for stimulating Fas-mediated apoptosis.

2. Description of Related Art

The Fas receptor, also known as APO-1 or CD95, is thought to be a key initiator of apoptotic programmed cell death in a variety of cell types. Activation of the Fas receptor by Fas ligands (FasL) or agonist antibodies lead to aggregation of the Fas receptor and recruitment of the intracellular death-inducing signal complex (DISC). See, for example, Kischkel et al., EMBO J. 14:5579-5588 (1995). Recruitment of other molecules, such as caspases, and in some cells, bcl-2, may also occur. It has been suggested that the main function of the Fas signaling complex is to activate caspase-8 protease. See, for example, Siegel and Fleischer, J. Allergy Clin. Immunol. 103:729 (1999). CD4⁺ T cells are unique in their ability to commit suicide by stimulating their own Fas receptors. T cells can also trigger apoptosis in B cells, macrophages, and other cell types through FasL. These interactions negatively regulate the immune system but can also contribute to immunopathology, as concerns Fas-mediated damage of target tissues in hepatitis and other organ specific autoimmune diseases. Fas plays a significant role in regulation of the human immune response, and the details of its clinical importance is being actively investigated. Altered Fas receptor or altered FasL are thought to contribute to autoimmune, infectious, and malignant diseases including autoimmune lymphoproliferative syndrome, autoimmune thyroid disease, hypereosinophilia, viral hepatitis, colon carcinomas, breast carcinomas, prostate cancers, neuroblastomas, and gliomas. See, for example, Houghton, J. Curr. Opin. Oncol. 11:475 (1999) and Siegel et al., J. Allergy Clin. Immunol. 103:729 (1999).

Acridines and analogs are known, and have been used as biological probes and potential therapies for many years. Acridines and acridine derivatives have been reported as potassium channel blockers (WO 98/54148), antihypertensives (EP 446604), antiviral agents (WO 97/27179, WO 96/39818, DE 444045, JP 01-221364), and monomeric units for dendrimers (WO 95/02397). Some compounds have been studied as potential anticancer and antitumor agents (U.S. Pat. No. 5,294,715, WO 99/58126, JP 01-221364). Other analogs have been reported to specifically targeted towards angiogenesis (WO 99/58126), topoisomerase (WO 99/64054), and p53 (WO 00/32175). No currently available literature is available on the action of acridines towards Fas-mediated apoptosis.

These and other documents referred to elsewhere in this application are incorporated herein by reference.

### SUMMARY OF THE INVENTION

In a first aspect, this invention includes methods of treating autoimmune and hyperplasic diseases in mammals, by administering to the mammal a therapeutically effective amount of a compound of the formula:

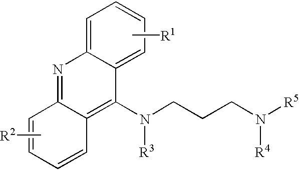

where:

R¹ and R² are independently selected from hydrogen, halogen, hydroxy, optionally substituted alkyl, optionally substituted alkyloxy, —NRR′ (where R is hydrogen or alkyl and R′ is hydrogen, alkyl, or aryl), and optionally substituted aryl; and

R₃, R₄, and R₅ are independently selected from hydrogen, optionally substituted alkyl, optionally substituted aryl, optionally substituted alkylcarbonyl, and optionally substituted arylcarbonyl, as a single stereoisomer or mixture of stereoisomers, or a pharmaceutically acceptable salt thereof.

The compounds of the invention stimulate Fas-mediated apoptosis, leading to cell death in cells containing the Fas receptor, and are useful in the regulation of the immune system and immune responses, and in hyperplasias (cell hyperproliferation). Thus, this invention is directed to methods for the treatment of autoimmune and hyperplasic diseases, particularly including autoimmune lymphoproliferative syndrome, autoimmune thyroid disease, hypereosinophilia, and the like. Optionally, the methods of treatment may also comprise administering another drug, such as a conventional form of therapy for the disease, to the mammal.

In a second aspect, this invention includes methods of stimulating Fas-mediated apoptosis in cells that have a Fas receptor. This method of stimulating Fas-mediated apoptosis in a cell that has a Fas receptor comprises contacting the cell with a compound of the formula:

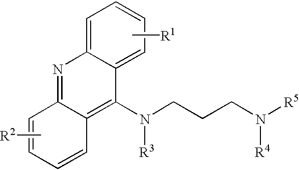

where:

R¹ and R² are independently selected from hydrogen, halogen, hydroxy, optionally substituted alkyl, optionally substituted alkyloxy, —NRR′ (where R is hydrogen or alkyl and R′ is hydrogen, alkyl, or aryl), and optionally substituted aryl; and

R₃, R₄, and R₅ are independently selected from hydrogen, optionally substituted alkyl, optionally substituted aryl, optionally substituted alkylcarbonyl, and optionally substituted arylcarbonyl, as a single stereoisomer or mixture of stereoisomers,

or a pharmaceutically acceptable salt thereof,

in an amount sufficient to stimulate Fas-mediated apoptosis. In vivo, the step of contacting the cells with such a compound may be effected by administering to an animal containing the cells an effective amount of the compound. In vitro, the step of contacting the cells with such a compound may be effected by administering an effective amount of the compound to the cells or to a solution bathing the cells.

In other aspects, this invention includes processes for identifying compounds that have at least one function selected from stimulating the Fas receptor and stimulating Fas-mediated apoptosis, identifying target compounds that mimic the function of the compounds used in the first aspect of this invention, and validating, optimizing, or standardizing bioassays, comprising the use of the compounds used in the first aspect of this invention.

### DETAILED DESCRIPTION OF THE INVENTION

(a) Definitions

“Alkyl” means a C₁-C₁₀ monovalent hydrocarbyl that may be linear, branched, or cyclic, and includes, for example, methyl, ethyl, propyl, isopropyl, butyl, isobutyl, sec-butyl, tert-butyl, pentyl, hexyl, cyclopentyl, cyclopropylmethyl, cyclohexyl, and cyclohexylmethyl. C₁-C₆ alkyls are preferred.

A “substituted alkyl” is an alkyl substituted with up to three halogen atoms and/or a substituent selected from —CN, —NO₂, —OR, —SR, —COR, —OC(O)R, —C(O)OR, —NR₂, —SO₂OR, —OSO₂R, —SO₂NR₂, —NRSO₂R, —CONR₂, and —NRCOR, where each R is, independently, hydrogen, optionally R′-substituted alkyl, optionally R′-substituted aryl, or optionally R′-substituted aralkyl, and each R′ is, independently, halo, —CN, —NO₂, —OH, C₁₋₃ alkyl, C₁₋₃ alkoxy, —SH, or —NH₂. Preferred substituted lower alkyls are substituted with up to three halogen atoms and/or one of the substituents selected from the group consisting of —CN, —NO₂, —OH, C₁₋₃ alkoxy, —SH, and —NH₂; and a particularly preferred substituted lower alkyl is —CF₃.

“Aryl” means an aromatic hydrocarbyl containing 6 to 20 ring carbon atoms, which is monocyclic (phenyl), condensed polycyclic, preferably condensed bicyclic (e.g., naphthyl), or linked polycyclic, preferably linked bicyclic (e.g., biphenylyl). The aryl is preferably C₆-C₁₆ and even more preferably, C₆-C₁₄. A particularly preferred aryl is phenyl.

A “substituted aryl” is an aryl substituted with up to three substituents selected from halo, —CN, —NO₂, —OR, optionally halo-substituted C₁₋₃ alkyl, optionally halo-substituted C₁₋₃ alkoxy, —SR, —COR, —OC(O)R, —C(O)OR, —NR₂, —SO₂OR, —OSO₂R, —SO₂NR₂, —NRSO₂R, —CONR₂, or —NRCOR, where each R is, independently, hydrogen or optionally R′-substituted alkyl and each R′ is, independently, halo, —CN, —NO₂, —OH, C₁₋₃ alkyl, C₁₋₃ alkoxy, —SH, or —NH₂. Preferred substituted aryls are substituted with up to three substituents selected from the group consisting of halo, —CN, —NO₂, —OH, optionally halo-substituted C₁₋₃ alkyl, optionally halo-substituted C₁₋₃ alkoxy, —SH, and —NH₂; and particularly preferred substituted aryls are substituted phenyls.

“Aralkyl” means an alkyl substituted with an aryl. Preferred aralkyls are benzyl and phenethyl.

A “substituted aralkyl” is an aralkyl in which the aryl or the alkyl, or both, are substituted in the manner described above for substituted aryl and substituted alkyl.

“Halogen” or “halo” means F, Cl, or Br.

“Pharmaceutically acceptable salts” are described in the section entitled “Compounds”.

A “therapeutically effective amount” means that amount which, when administered to an animal for treating a disease, is sufficient to effect such treatment for the disease.

“Treating” or “treatment” of a disease in a mammal includes (1) preventing the disease from occurring in a mammal which may be predisposed to the disease but does not yet experience or display symptoms of the disease, (2) inhibiting the disease, i.e., arresting its development, (3) relieving symptoms of the disease, i.e., reducing the effects of the disease, and (4) causing regression of the disease.

(b) Compounds

The compounds useful in this invention are compounds of the formula:

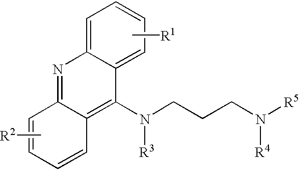

where:

R¹ and R² are independently selected from hydrogen, halogen, hydroxy, optionally substituted alkyl, optionally substituted alkyloxy, —NRR′ (where R is hydrogen or alkyl and R′ is hydrogen, alkyl, or aryl), and optionally substituted aryl; and

R₃, R₄, and R₅ are independently selected from hydrogen, optionally substituted alkyl, optionally substituted aryl, optionally substituted alkylcarbonyl, and optionally substituted arylcarbonyl, as a single stereoisomer or mixture of stereoisomers, and the pharmaceutically acceptable salts thereof.

Preferred compounds are those where R¹ and R² are hydrogen. More preferred compounds are those where R¹, R², and R³ are hydrogen. Especially preferred compounds are those where R¹, R², and R³ are hydrogen, and R⁴ and R⁵ are alkyl. The most preferred compound is that where R¹, R², and R³ are hydrogen, and R⁴ and R⁵ are ethyl, i.e. the compound 9-[(3-diethylaminopropyl)amino]acridine.

Syntheses and descriptions of these compounds are outlined in the Examples.

Certain compounds of the invention may contain one or more chiral centers. In such cases, all stereoisomers also fall within the scope of this invention. The invention compounds include the individually isolated stereoisomers as well as mixtures of such stereoisomers.

Pharmaceutically acceptable salts, cations and anions of the compounds of the invention are also included in the present invention and are useful in the methods described herein.

Pharmaceutically acceptable salts include salts which may be formed when acidic protons present are capable of reacting with inorganic or organic bases. Typically the parent compound is treated with an excess of an alkaline reagent, such as hydroxide, carbonate or alkoxide, containing an appropriate cation. Cations such as Na⁺, K⁺, Ca²⁺, Mg²⁺ and NH₄⁺ are examples of cations present in pharmaceutically acceptable salts. The Na⁺ salts are especially useful. Acceptable inorganic bases, therefore, include aluminum hydroxide, calcium hydroxide, potassium hydroxide, sodium carbonate and sodium hydroxide. Salts may also be prepared using organic bases, such as salts of primary, secondary and tertiary amines, substituted amines including naturally-occurring substituted amines, and cyclic amines including isopropylamine, trimethylamine, diethylamine, triethylamine, tripropylamine, ethanolamine, 2-dimethylaminoethanol, tromethamine, lysine, arginine, histidine, caffeine, procaine, hydrabamine, choline, betaine, ethylenediamine, glucosamine, N-alkylglucamines, theobromine, purines, piperazine, piperidine, N-ethylpiperidine, and the like.

If a compound of this invention contains a basic group, an acid addition salt may be prepared. Acid addition salts of the compounds are prepared in a standard manner in a suitable solvent from the parent compound and an excess of acid, such as hydrochloric acid, hydrobromic acid, sulfuric acid (giving the sulfate and bisulfate salts), nitric acid, phosphoric acid and the like, and organic acids such as acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, salicylic acid, p-toluenesulfonic acid, hexanoic acid, heptanoic acid, cyclopentanepropionic acid, lactic acid, o-(4-hydroxy-benzoyl)benzoic acid, 1,2-ethanedisulfonic acid, 2-hydroxyethanesulfonic acid, benzenesulfonic acid, p-chlorobenzenesulfonic acid, 2-naphthalenesulfonic acid, camphorsulfonic acid, 4-methyl-bicyclo[2.2.2.]oct-2-ene-1-carboxylic acid, glucoheptonic acid, gluconic acid, 4,4′-methylenebis(3-hydroxy-2-naphthoic)acid, 3-phenylpropionic acid, trnmethylacetic acid, t-butylacetic acid, laurylsulfuric acid, glucuronic acid, glutamic acid, 3-hydroxy-2-naphthoic acid, stearic acid, muconic acid and the like.

Certain of the compounds may form inner salts or Zwitterions.

(c) Pharmaceutical Compositions

The methods of this invention may be practiced by the administration of the compounds themselves or, particularly in the first aspect of this invention, by the administration of the compounds in pharmaceutical compositions comprising a compound useful in the first aspect of this invention and a pharmaceutically acceptable excipient.

The pharmaceutical compositions of the invention preferably comprise as an active ingredient a preferred compound of the first aspect of this invention. However, pharmaceutical compositions that comprise any of the compounds of the invention are contemplated. The pharmaceutical compositions of the invention also comprise a pharmaceutically acceptable excipient.

The compositions of this invention may be administered by any number of routes, including but not limited to, intravenous, intramuscular, intra-arterial, intramedullary, intrathecal, intraventricular, transmucosal or transdermal, subcutaneous, intraperitoneal, intranasal, enteral, sublingual, rectally or by way of other body cavity, including suppository and the like, topical, or may be administered orally. Administration may be acute, or by means of controlled-release, slow release or sustained release systems, including orally-administered time-release capsules or other delivery means, depot administration, indwelling catheter, chronic administration via a transdermal drug-delivery patch or subdermal implant, such that a relatively constant level of dosage is maintained. See, e.g., U.S. Pat. No. 3,710,795.

Formulations may be aqueous, oily, emulsified, or contain solvents suitable to the mode of administration, and may optionally be liposomal formulations, formulations designed to administer the drug across mucosal membranes or transdermal formulations. Suitable formulations for each of these and other methods of administration discussed in this application may be found, for example, in Gennaro, ed., “Remington: The Science and Practice of Pharmacy”, 20th ed., 2000, Lippincott, Williams & Wilkins, Philadelphia Pa.

Depending on the intended mode of administration, the pharmaceutical compositions may be in the form of solid, semi-solid or liquid dosage forms, such as, for example, tablets, suppositories, pills, capsules, powders, liquids, suspensions, creams, ointments, lotions or the like, preferably in unit dosage form suitable for single administration of a precise dosage. In addition to an effective amount of the active ingredients, the compositions may contain suitable pharmaceutically-acceptable excipients and auxiliaries which facilitate processing of the active compounds into preparations which can be used pharmaceutically. As used herein, the term “pharmaceutically acceptable excipient” refers to an excipient or mixture of excipients which does not interfere with the effectiveness of the biological activity of the active ingredients and which is not toxic to the host to which it is administered.

In addition, the pharmaceutical compositions may include other pharmaceutical agents, adjuvants, diluents, buffers, etc. The compounds may thus be administered orally, parenterally, transdermally, rectally, nasally, buccally, topically or via an implanted reservoir in dosage formulations containing conventional non-toxic pharmacologically acceptable carriers, adjuvants and vehicles. The term “parenteral” as used herein is intended to include subcutaneous, intravenous, and intramuscular injection.

For solid compositions, conventional nontoxic solid carriers include, for example, pharmaceutical grades of mannitol, lactose, starch, magnesium stearate, sodium saccharin, talc, cellulose, glucose, sucrose, magnesium carbonate, and the like. Liquid pharmacologically administrable compositions can, for example, be prepared by dissolving, dispersing, etc., an active compound as described herein and optional pharmaceutical adjuvants in an excipient, such as, for example, water, saline, aqueous dextrose, glycerol, ethanol, and the like, to thereby form a solution or suspension. If desired, the pharmaceutical composition to be administered may also contain minor amounts of nontoxic auxiliary substances such as wetting or emulsifying agents, pH buffering agents and the like, for example, sodium acetate, sorbitan monolaurate, triethanolamine sodium acetate, triethanolamine oleate, etc.

For oral administration, the composition will generally take the form of a tablet or capsule, or it may be an aqueous or nonaqueous solution, suspension or syrup. Tablets and capsules are preferred oral administration forms. Tablets and capsules for oral use will generally include one or more commonly used carriers such as lactose and corn starch. Lubricating agents, such as magnesium stearate, are also typically added. When liquid suspensions are used, the active agent may be combined with emulsifying and suspending agents. If desired, flavoring, coloring and/or sweetening agents may be added as well. Other optional components for incorporation into an oral formulation herein include, but are not limited to, preservatives, suspending agents, thickening agents, and the like.

Parenteral administration, if used, is generally characterized by injection. Injectable formulations can be prepared in conventional forms, either as liquid solutions or suspensions, solid forms suitable for solubilization or suspension in liquid prior to injection, or as emulsions. Preferably, sterile injectable suspensions are formulated according to techniques known in the art using suitable carriers, dispersing or wetting agents and suspending agents. The sterile injectable formulation may also be a sterile injectable solution or a suspension in a nontoxic parenterally acceptable diluent or solvent. Among the acceptable vehicles and solvents that may be employed are water, Ringer's solution and isotonic sodium chloride solution. In addition, sterile, fixed oils, fatty esters or polyols are conventionally employed as solvents or suspending media.

The compounds of the invention may also be delivered through the skin using conventional transdermal drug delivery systems, i.e., transdermal “patches” wherein the agent is typically contained within a laminated structure that serves as a drug delivery device to be affixed to the skin. In such a structure, the drug composition is typically contained in a layer, or “reservoir,” underlying an upper backing layer. The laminated device may contain a single reservoir, or it may contain multiple reservoirs. In one embodiment, the reservoir comprises a polymeric matrix of a pharmaceutically acceptable contact adhesive material that serves to affix the system to the skin during drug delivery. Examples of suitable skin contact adhesive materials include, but are not limited to, polyethylenes, polysiloxanes, polyisobutylenes, polyacrylates, polyurethanes, and the like. Alternatively, the drug-containing reservoir and skin contact adhesive are present as separate and distinct layers, with the adhesive underlying the reservoir which, in this case, may be either a polymeric matrix as described above, or it may be a liquid or hydrogel reservoir, or may take some other form.

Alternatively, the pharmaceutical compositions of the invention may be administered in the form of suppositories for rectal administration. These can be prepared by mixing the agent with a suitable non-irritating excipient which is solid at room temperature but liquid at the rectal temperature and therefore will melt in the rectum to release the drug. Such materials include cocoa butter, beeswax and polyethylene glycols.

The pharmaceutical compositions of the invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, propellants such as fluorocarbons or nitrogen, and/or other conventional solubilizing or dispersing agents.

Preferred formulations for topical drug delivery are ointments and creams. Ointments are semisolid preparations which are typically based on petrolatum or other petroleum derivatives. Creams containing the selected active agent are, as known in the art, viscous liquid or semisolid emulsions, either oil-in-water or water-in-oil. Cream bases are water-washable, and contain an oil phase, an emulsifier and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation is generally a nonionic, anionic, cationic or amphoteric surfactant. The specific ointment or cream base to be used, as will be appreciated by those skilled in the art, is one that will provide for optimum drug delivery. As with other carriers or vehicles, an ointment base should be inert, stable, nonirritating and nonsensitizing.

Formulations for buccal administration include tablets, lozenges, gels and the like. Alternatively, buccal administration can be effected using a transmucosal delivery system as known to those skilled in the art.

The pharmaceutical compositions of this invention may be formulated as solutions or lyophilized powders for parenteral administration. Powders may be reconstituted by addition of a suitable diluent or other pharmaceutically acceptable carrier prior to use. The liquid formulation is generally a buffered, isotonic, aqueous solution. Examples of suitable diluents are normal isotonic saline solution, 5% dextrose in water or buffered sodium or ammonium acetate solution. Such formulations are especially suitable for parenteral administration, but may also be used for oral administration. It may be desirable to add excipients such as polyvinylpyrrolidinone, gelatin, hydroxycellulose, acacia, polyethylene glycol, mannitol, sodium chloride or sodium citrate. Alternatively, these compounds may be encapsulated, tableted, or prepared in an emulsion or syrup for oral administration. Pharmaceutically acceptable solid or liquid excipients may be added to enhance or stabilize the composition, or to facilitate preparation of the composition. Liquid excipients include syrup, peanut oil, olive oil, glycerin, saline, alcohol, and water. Solid excipients include starch, lactose, calcium sulfate dihydrate, terra alba, magnesium stearate, stearic acid, talc, pectin, acacia, agar, and gelatin. The excipient may also include a sustained release material such as glyceryl monostearate or glyceryl distearate, alone or with a wax. The amount of solid excipient varies but, preferably, will be between about 20 mg to about 1 g per dosage unit. The pharmaceutical preparations are made following the conventional techniques of pharmacy involving milling, mixing, granulation, and compressing, when necessary, for tablet forms; or milling, mixing and filling for hard gelatin capsule forms. When a liquid excipient is used, the preparation will be in the form of syrup, elixir, emulsion or aqueous or non-aqueous suspension. Such a liquid formulation may be administered orally directly or filled into a soft gelatin capsule.

The pharmaceutical formulation may additionally contain one or more pharmacologically active agents in addition to a compound of the invention. These additional active agents will typically be useful for preventing or treating autoimmune, infectious and malignancies, or for enhancing the treatment of such disorders by compounds of the invention.

Some specific examples of suitable pharmaceutical compositions are described in the Examples below.

Typically, a pharmaceutical composition of the present invention is packaged in a container with a label, or instructions, or both, indicating use of the pharmaceutical composition in the treatment of autoimmune and hyperplasic diseases, such as autoimmune lymphoproliferative syndrome, autoimmune thyroid disease, hypereosinophilia, and the like.

(c) Methods and Uses of Compounds of this Invention.

The compounds of the invention are effective to stimulate Fas-mediated apoptosis as demonstrated in the Examples below. Stimulation of Fas-mediated apoptosis is useful, for example, in the treatment of autoimmune and hyperplasic diseases, such as autoimmune lymphoproliferative syndrome, autoimmune thyroid disease, hypereosinophilia, and the like.

Thus, the first aspect of this invention includes a method of treating an autoimmune or hyperplasic disease in a mammal, preferably a human, by administering a therapeutically effective amount of a compound of this invention, or a pharmaceutical composition thereof, to the mammal. Optionally, the method may further comprise treating the mammal with a conventional form of therapy for an autoimmune or hyperplasic disease, such as administration of a conventional immunosuppressant or antihyperprolifertive agent. The total amount of the combination of drugs administered to the mammal must be a therapeutically effective amount, although the amounts of each of the individual drugs may be, by themselves, suboptimal for therapeutic purposes.

The compounds of the invention, or pharmaceutical compositions thereof, are thus used to stimulate Fas-mediated apoptosis in mammals that require such treatment, by administering a therapeutically effective amount of the chosen compound, preferably dispersed in a pharmaceutical carrier. Therapeutically effective amounts of compounds of the invention are in the range of 0.01 to 1000 mg/kg, preferably 0.01 to 100 mg/kg and more preferably 1 to 30 mg/kg, and suitable doses will be readily determined by one skilled in the art depending upon the route of administration, age and condition of the patient. The dosage units may be administered up to one to ten times daily for acute or chronic disease. No unacceptable toxicological effects are expected when compounds of the invention are administered in accordance with the present invention.

In another aspect of the invention, Fas-mediated apoptosis is stimulated by contacting a cell having a Fas receptor with a compound of the invention in an amount sufficient to stimulate Fas-mediated apoptosis. In such a case, the contacting is effected in vivo by administering the compound, or a pharmaceutical composition thereof, to a mammal containing the cell; and in vitro by administering the compound, or a pharmaceutical composition thereof, to a container in which the cell is present or to a solution bathing the cell.

The compounds of the invention have been demonstrated to stimulate Fas-mediated apoptosis and can be useful in the treatment of autoimmune and hyperplasic diseases. Similarly, other compounds which show the same effects on Fas-mediated apoptosis can be useful for the treatment of autoimmune and hyperplasic diseases. The compounds disclosed in this application can be used as models to discover other new agents that act to stimulate Fas-mediated apoptosis. The steps in a process in which these compounds can be utilized to discover new therapeutic agents may be achieved by the following: the compounds may be utilized to validate, optimize, and standardize assays necessary for the discovery of other compounds that stimulate Fas-mediated apoptosis and that stimulate Fas-mediated apoptosis by action at the Fas receptor. These compounds can be utilized as benchmarks to discover other agents that show improved activity in assays that:

1. activate/stimulate the Fas receptor;

2. block the Fas receptor;

3. stimulate Fas-mediated apoptosis;

4. affect Fas-mediated regulation of cell proliferation; and/or

5. affect Fas-mediated regulation of the immune response.

A method to discover agents that show improved activity in assays that activate/stimulate the Fas receptor, that block the Fas receptor, that stimulate Fas-mediated apoptosis, or that affect Fas-mediated regulation of cell proliferation and/or the immune response, comprises the steps of obtaining the results of an assay for Fas-mediated apoptosis in the presence of a plurality of concentrations of a compound of the invention, obtaining the results of the assay in the presence of a plurality of concentrations of a test compound, comparing the results of the assays, and identifying as an agent that shows improved activity in assays that measure or detect interaction with the Fas receptor, that stimulate Fas-mediated apoptosis, or that affect Fas-mediated regulation of cell proliferation and/or the immune response, a test compound from which the results obtained in the assay were improved compared to the results obtained with the compound of the invention.

Algorithms may be used to compare structures or chemical properties of compounds, such as exemplary compounds and other test compounds. Algorithms may also be used to match structures or chemical properties within libraries of test compounds. In this way, where exemplary compounds or test compounds are known to have certain structures, properties, or activities of interest, compounds can be utilized to discover other compounds or agents that also have such structures, properties, or activities. For example, an activity of interest may be a desired activity in a bioassay. Such algorithms are known; for example, U.S. Pat. No. 5,567,317 and U.S. Pat. No. 5,587,293 describe methods for determining the reactivity of candidate compounds with target moieties or receptors. A formula predictive of reactivity with the target receptor may be obtained from a reference set of receptors or from a panel of compounds that are systematically diverse with respect to certain properties. Compounds to be tested in this way can be physically assessed with respect to the reference receptors, the formula applied, and the expected reactivity with the actual target receptor may be predicted. The method of U.S. Pat. No. 5,587,293 does not require the physical presence of the receptor.

The use of such algorithms that compare structures or chemical properties and/or match structures or chemical properties within libraries of test compounds, is effective to discover agents that display activity in bioassays. Such bioassays include bioassays to detect and measure interaction with the Fas receptor, blockade of the Fas receptor, Fas-mediated apoptosis, activation of Fas-mediated apoptosis, stimulation of Fas-mediated apoptosis, and effects on Fas-mediated regulation of cell proliferation, and Fas-mediated regulation of the immune response.

In addition, when combined with algorithms that compare structures or chemical properties and/or match structures or chemical properties within libraries of test compounds, these compounds can be utilized to discover agents that display activity in bioassays that:

1. activate/stimulate the Fas receptor;

2. block the Fas receptor;

3. stimulate Fas-mediated apoptosis;

4. affect Fas-mediated regulation of cell proliferation; and/or

5. affect Fas-mediated regulation of the immune response.

A method to discover agents that display activity in bioassays that activate/stimulate the Fas receptor, that block the Fas receptor, that stimulate Fas-mediated apoptosis, or that affect Fas-mediated regulation of cell proliferation and/or the immune response, comprising applying an algorithm to compare the chemical structures or chemical properties within a library of test compounds with the chemical structure or chemical properties of a compound of the invention, and identifying as an agent that displays activity in bioassays that activate/stimulate the Fas receptor, that block the Fas receptor, that stimulate Fas-mediated apoptosis, or that affect Fas-mediated regulation of cell proliferation and/or the immune response, a test compound determined by the algorithm to have a chemical structure or chemical properties similar to the compound of the invention.

Algorithms may also be used to compare structures and/or match structures for the purpose of modeling molecular interactions. Such algorithms are known; for example, the methods of U.S. Pat. No. 5,567,317 and U.S. Pat. No. 5,587,293 may be used to compare structures and/or match structures for the purpose of modeling molecular interactions.

The use of such algorithms is effective to discover agents that display activity in bioassays such as bioassays to detect and measure interaction with the Fas receptor, blockade of the Fas receptor, Fas-mediated apoptosis, activation of Fas-mediated apoptosis, stimulation of Fas-mediated apoptosis, and effects on Fas-mediated regulation of cell proliferation, and Fas-mediated regulation of the immune response.

Further, when combined with algorithms that compare structures and/or match structures for the purpose of modeling molecular interactions, these compounds can be utilized to discover agents that display activity in bioassays that:

1. activate/stimulate the Fas receptor;

2. block the Fas receptor;

3. stimulate Fas-mediated apoptosis;

4. affect Fas-mediated regulation of cell proliferation; and/or

5. affect Fas-mediated regulation of the immune response.

A method to discover agents that display activity in bioassays that activate/stimulate the Fas receptor, that block the Fas receptor, that stimulate Fas-mediated apoptosis, or that affect Fas-mediated regulation of cell proliferation and/or the immune response, comprising applying an algorithm to compare and/or match the chemical structures within a library of test compounds with the chemical structure of a compound of the invention for the purpose of modeling molecular interactions, and identifying as an agent that activates/stimulates the Fas receptor, that blocks the Fas receptor, that stimulates Fas-mediated apoptosis, or that affects Fas-mediated regulation of cell proliferation and/or the immune response, a test compound determined by the algorithm to have chemical structure comparable to or matching the compound of the invention.

In addition, the methods of the invention include a process for validating, optimizing, or standardizing a bioassay. This process comprises (a) submitting a compound of the invention to the bioassay; and (b), validating, optimizing, or standardizing the bioassay by the activity of the compound in the bioassay.

### EXAMPLES

The following Examples illustrate this invention, and are in no way intended to limit the scope of this invention.

The compounds of this invention are prepared by conventional methods of organic chemistry, and many methods for the synthesis of substituted acridines are well known to the art. See, for example, Larock, “Comprehensive Organic Transformations”, Wiley-VCH, New York N.Y. In some cases, protective groups may be introduced and later removed. Suitable protective groups for amino, hydroxyl, and carboxyl groups are described in Greene et al. “Protective Groups in Organic Synthesis”, 2nd ed., 1991, John Wiley and Sons, New York N.Y. The compounds of this invention can be synthesized as shown in the following examples or by modifying the exemplified syntheses by means known to those of ordinary skill in the art.

A typical synthesis is shown in Reaction Scheme 1 below. The reaction of an optionally substituted 9-chloroacridine [or other acridine where the 9-position is substituted with a leaving group such as an alkane- or arenesulfonate] with a substituted 1,3-diaminopropane affords the 9-(substituted propylamino)acridine in high yield.

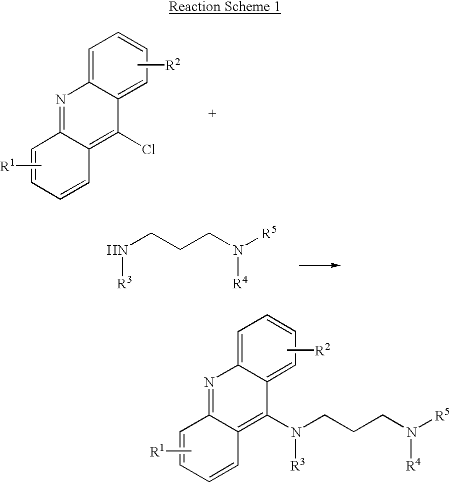

Variation of the acridine and the 1,3-diaminopropane affords a variety of the compounds of this invention.

### Example 1

### 9-[(3-diethylaminopropyl)amino]acridine

N,N-diethyl-1,3-propanediamine (0.2 g, 1.5 mmol) was added to a solution of 9-chloro-acridine (0.21 g, 1 mmol) in dimethylformamide, and the reaction mixture was heated to 80° C. for 48 hours, After cooling, the mixture was partitioned between aqueous sodium bicarbonate and dichloromethane solutions were washed the aqueous layer extracted three times with dichloromethane. The combined dichloromethane solutions were washed with aqueous sodium bicarbonate and dried over potassium carbonate. The crude oil was chromatographed to afford 9-[(3-diethylamino-propyl)amino]acridine, compound 3 in the table below (0.12 g, 38% yield).

The table below gives representative compounds of this invention prepared by the method of Example 1.

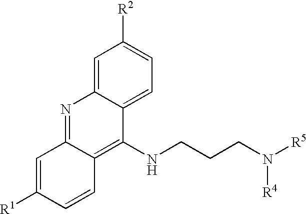

| Compound | R¹ | R² | R⁴ | R⁵ |
|---|---|---|---|---|
| 3 | H | H | ethyl | ethyl |
| 7 | H | H | methyl | methyl |
| 8 | Cl | Cl | methyl | methyl |
| 9 | Cl | Cl | ethyl | ethyl |
| 10 | H | H | H | methyl |
| 11 | H | H | H | ethyl |
| 12 | H | H | H | benzyl |
| 13 | H | H | benzyl | benzyl |
| 14 | Cl | Cl | benzyl | benzyl |
| 15 | H | H | H | H |

Other acridines may be prepared by preparation of an acridine having an incompletely substituted propylamine side chain followed by acylation of that side chain as shown in Reaction Scheme 2.

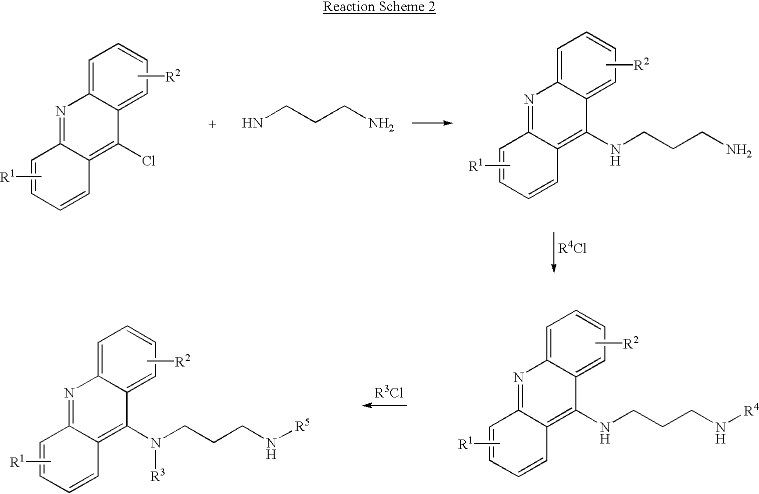

In this Reaction Scheme 2, each of R³Cl and R⁴Cl are acyl chlorides [or other acyl halides, or similar activated acid derivatives], so that the resulting compound is an acridine with a mono- (if the acridine is reacted only with R³Cl) or di-acylated (if the acridine is reacted in turn with R³Cl and R⁴Cl) propylamine side chain. Variation of the starting acridine and the 1,3-diaminopropane, together with variation in the acyl halides, affords a variety of the compounds of this invention.

### Example 2

### 9-[(3-Acetylaminopropyl)(benzoyl)amino]acridine

To a solution of 9-[(3-aminopropyl)amino]acridine (0.25 g, 1 mmol), compound 15 in the table above, prepared by the method of Example 1, in dichloromethane at 0° C. was added a solution of acetyl chloride (0.078 g, 1 mmol) in dichloromethane. The reaction mixture was stirred for 2 hours, and then diluted with water. The mixture was neutralized with aqueous sodium bicarbonate and the dichloromethane layer separated. The aqueous layer was extracted with dichloromethane. The combined dichloromethane solution was washed with brine and dried over potassium carbonate, and concentrated under vacuum to a yellow oil. The crude oil was redissolved in dichloromethane, and a solution of benzoyl chloride (0.140 g, 1 mmol) was added. The mixture was stirred for 24 hours at room temperature, then neutralized with aqueous sodium bicarbonate, and the dichloromethane layer separated, and the aqueous layer extracted with dicloromethane. The combined dichloromethane solution was washed with brine and dried over potassium carbonate, then concentrated under vacuum to afford 9-[(3-acetylaminopropyl)-(benzoyl)amino]acridine, compound 16 in the table below, as a colorless oil (0.160 g, 44% yield).

The table below gives representative compounds of this invention prepared by the method of Example 2.

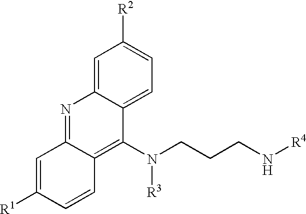

| Compound | R¹ | R² | R³ | R⁴ |
|---|---|---|---|---|
| 16 | H | H | benzoyl | acetyl |
| 17 | H | H | H | acetyl |
| 19 | H | H | H | benzoyl |
| 20 | H | H | acetyl | benzoyl |
| 21 | H | H | benzoyl | benzoyl |
| 22 | H | H | propionyl | propionyl |
| 23 | H | H | acetyl | 1-naphthylcarbonyl |
| 24 | H | H | 1-naphthylcarbonyl | acetyl |
| 25 | H | H | H | 1-naphthylcarbonyl |
| 26 | H | H | H | isobutyryl |
| 27 | H | H | isobutyryl | isobutyryl |
| 27 | H | H | isobutyryl | acetyl |
| 28 | H | H | acetyl | isobutyryl |
| 29 | H | H | H | isovaleryl |
| 30 | H | H | isovaleryl | isovaleryl |
| 31 | H | H | acetyl | isovaleryl |
| 32 | H | H | isovaleryl | acetyl |
| 33 | H | H | 4-biphenylylcarbonyl | 4-biphenylylcarbonyl |
| 34 | H | H | H | 4-biphenylylcarbonyl |
| 35 | H | H | acetyl | 4-biphenylylcarbonyl |
| 36 | H | H | 4-biphenylylcarbonyl | acetyl |
| 37 | H | H | 3-methoxypropionyl | 3-methoxypropionyl |
| 38 | H | H | acetyl | 3-methoxypropionyl |
| 39 | H | H | 3-methoxypropionyl | acetyl |
| 40 | Cl | Cl | acetyl | acetyl |
| 41 | Cl | Cl | benzoyl | benzoyl |
| 42 | Cl | Cl | acetyl | benzoyl |

### Example 3

### Fas-Mediated Apoptosis

Assays for Fas-dependent apoptosis are known in the art. See, for example, Ruiz-Ruiz et al, Cell Death Diff. 6:271 (1999); and Muller et al., J. Exp. Therap. 188:2033 (1998).

Apoptosis is identified by detection of the DNA fragmentation pattern characteristic of apoptotic cell death. In this Example, apoptosis is detected and measured by FACS® analysis carried out in a FACScan® flow cytometer (Becton Dickinson) using CellQuest software. Quantification of DNA fragmentation is performed by FACS® analysis of propidium iodide-stained nuclei as described in Nicolletti et al., J. Immunol. Methods 139:271-279 (1991). Hepatocytes floating in the culture medium are collected by centrifugation at 200× g. Adherent hepatocytes are harvested by incubation with 1% trypsin for 1 min. The cells are washed in phosphate-buffered saline (PBS), suspended in hypotonic lysis buffer (0.1% sodium citrate, 0.1% Triton X, and 50 ng/mL propidium iodide) (Sigma) and incubated at 4° C. for 6 hours. Cells are then analyzed for DNA content by flow cytometry.

Early apoptotic changes are identified using annexin V-Fluos (Boehringer Mannheim) which binds to phosphatidylserine molecules (PS) exposed on apoptotic, but not normal, cell membranes (PS is normally restricted to the inner leaflet of the cell membrane bilayer). Propidium iodide is used to discriminate necrotic cells from the annexin V positively stained cell cluster. Cells are typsinized, washed with PBS, centrifuged at 200× g for 5 min, and resuspended in 100 μL HEPES buffer (10 ml HEPES/NaOH, pH 7.4, 140 mM NaCl, 5 mM CaCl₂) and 20 μL propidium iodide. Cells are incubated for 10-15 min and analyzed on a flow cytometer using CellQuest software. A 488 nm excitation wavelength and a 560 nm cutoff filter is used for detection of propidium iodide.

Compound 3 shows stimulation of Fas-dependent apoptosis at 10 μM.

### Example 4

### Stimulation of Fas-Mediated Apoptosis

Anti-human Fas antibody h-HFE7A, humanized antibody was obtained from Sankyo Co., Ltd. This antibody induces apoptosis when cross-linked with secondary antibody in vitro.

Samples of human synovium were obtained from rheumatoid arthritis patients at the time of total knee replacement surgery or synoviectomy. Synovial tissue was minced into small pieces and was digested with collagenase and cultured in Ham's F12 medium supplemented with 10% fetal bovine serum (F10F) in a humidified 5% CO₂ atmosphere at 37° C. Adherent cells were considered as synoviocytes and were cultivated in F10F.

Cell viability was determined with the XTT method described in Cancer Res. 48:4827 (1988). Ninety-six well flat plates were precoated with anti-human IgG. h-HFE7A (1000 ng/mL) and/or 10 μM of a compound of this invention were added and incubated for 2 hours. Synoviocytes were seeded (10,000/well) and incubated for 16 hours. Background wells received culture medium only. XTT (2,3-Bis[2-methoxy-4-nitro-sulfophenyl]-2H-tetrazolium-5-carboxanilide), final concentration 200 μg/mL, and phenazine methosulfate, final concentration 5 μM, were added to each well, and further incubated for 4 hours. The absorbance at 450 nm was measured and cell viability was determined as follows:

$$Cell Viability ( \% ) = \frac{( {OD}_{450} of test sample - {OD}_{450} of background ) \times 100}{( {OD}_{450} of control sample - {OD}_{450} of background )}$$

Enhancement of Fas-mediated apoptosis by compounds of this invention was determined by the stimulation index (SI):

$$SI = \frac{\begin{matrix}( Cell viability ( \% ) with compound only ) \times \\ ( Cell viability ( \% ) with h \text{-} HFE7A only )\end{matrix}}{( Cell viability ( \% ) with compound and h \text{-} HEF7A \times 100}$$

An SI over 2.0 was considered positive.

Compound 3 was positive in this assay at 10 μM.

### Example 5

### Expression of the Fas Receptor

Expression of the Fas receptor is measured by the method of Muller et al., J. Exp. Med. 188:2033-2045 (1998). A FACScan® flow cytometer (Becton Dickinson) using CellQuest software is used to determine the percent enhanced Fas receptor expression. The antibody anti-APO-1 (IgG3), specific for the Fas receptor, is used as a purified biotinylated antibody. Quantum Red streptavidin (Sigma) is used as a secondary reagent for indirect immunofluorescence. Hepatoma cells are incubated in 50 μL culture medium with biotinylated anti-APO-1. After 30 min incubation, cells are washed twice, incubated for 30 min with Quantum Red streptavidin, washed twice again, and assayed. Upon data acquisition, a gate is set on intact cells by forward/side scatter analysis, and 1 viable cells are analyzed. The percent enhanced Fas receptor expression is calculated as the difference between the % Fas receptor detected in treated cells and the % Fas receptor detected in control cells, according to the formula:

Enhanced Fas receptor expression (%)=(% Fas receptor in treated cells−% Quantum Red in treated cells)−(% Fas receptor in control cells−% Quantum Red in control cells).

Compounds of the invention are found to enhance Fas receptor expression.

### Example 6

### Oral Pharmaceutical Composition Preparation—Solid Dosage Formulation

A pharmaceutical composition for oral administration is prepared by combining the following:

| | % w/w |
|---|---|
| Compound of the invention | 10% |
| Magnesium stearate | 0.5% |
| Starch | 2.0% |
| hydroxypropylmethylcellulose | 1.0% |
| Microcrystalline cellulose | 86.5% |

The mixture is compressed in a press to form tablets. Alternatively, , the mixture is instead filled into hard gelatin capsules.

Tablets may be coated by applying a suspension of film former (e.g. hydroxypropyl-methylcellulose), pigment (e.g. titanium dioxide) and plasticizer (e.g. diethyl phthalate) and drying the film by evaporation of the solvent. The film coat is typically between 2% and 6% of the tablet by weight, e.g. 3% by weight.

Tablets comprising compounds of the invention made by the methods of this Example are suitable for oral administration and are effective in the enhancement of Fas-mediated apoptosis and for the treatment of autoimmune diseases, infectious diseases, and malignancies.

### Example 7

### Oral Pharmaceutical Composition Preparation—Softgel

A pharmaceutical composition of a compound of the invention suitable for oral administration is prepared by combining the following:

| | % w/w |
|---|---|
| Compound of the invention | 20% |
| Polyethylene glycol | 80% |

The compound is dispersed or dissolved in the liquid carrier, and a thickening agent is optionally added. The formulation is then enclosed in a soft gelatin capsule.

Soft gelatin capsules comprising compounds of the invention made by the methods of this example are suitable for oral administration and are effective in the enhancement of Fas-mediated apoptosis and for the treatment of autoimmune diseases, infectious diseases, and malignancies.

### Example 8

### Pharmaceutical Composition for Parenteral Administration

Pharmaceutical compositions for parenteral administration typically comprise the pharmaceutically active ingredient and physiological saline, such as phosphate buffered saline or other water solution with pH and salt content suitable for introduction into an animal. A pharmaceutical composition for parenteral administration is prepared by combining a compound of the invention and Dulbecco's Phosphate Buffered Saline (D8662, Sigma Chemical Co. St. Louis Mo.) as described in the following:

| | % w/w |
|---|---|
| Compound of the invention | 1.0% |
| Saline | 99.0% |

The solution is sterilized and sealed in sterile containers.

Pharmaceutical compositions comprising compounds of the invention made by the methods of this example are suitable for parenteral administration and are effective in the enhancement of Fas-mediated apoptosis and for the treatment of autoimmune diseases, infectious diseases, and malignancies.

Various modifications and variations of the present invention will be apparent to those skilled in the art without departing from the scope and spirit of the invention. Although the invention has been described in connection with specific preferred embodiments, it should be understood that the invention as claimed is not limited to such specific embodiments. It will be appreciated by one of ordinary skill in the art that various modifications of the described modes for carrying out the invention are within the scope of the following claims.

We claim:

1. A method of treating an autoimmune or hyperplasic disease in a mammal, comprising administering to the mammal a therapeutically effective amount of a compound of the formula: 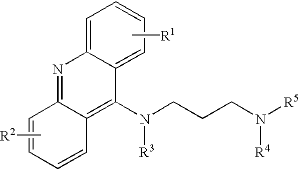
where:
R¹ and R² are independently selected from hydrogen, halogen, hydroxy, optionally substituted alkyl, optionally substituted alkyloxy, —NRR′ (where R is hydrogen or alkyl and R′ is hydrogen, alkyl, or aryl), and optionally substituted aryl; and
R₃, R₄, and R₅ are independently selected from hydrogen, optionally substituted alkyl, optionally substituted aryl, optionally substituted alkylcarbonyl, and optionally substituted arylcarbonyl, as a single stereoisomer or mixture of stereoisomers, or a pharmaceutically acceptable salt thereof.

2. The method of claim 1, where R¹ and R² are hydrogen.

3. The method of claim 2, where R³ is hydrogen.

4. The method of claim 3, where R⁴ and R⁵ are alkyl.

5. The method of claim 4, where the compound is 9-[(3-diethylaminopropyl)amino]acridine or a pharmaceutically acceptable salt thereof.

6. The method of claim 1, where the disease is an autoimmune disease.

7. The method of claim 1, where the disease is a hyperplasic disease.

8. The method of claim 1, where the disease is autoimmune lymphoproliferative syndrome, autoimmune thyroid disease, or hypereosinophilia.

9. The method of claim 1, further comprising treating said mammal with an additional form of therapy for said disease state.

10. A method of stimulating Fas-mediated apoptosis in a cell having a Fas receptor, comprising contacting the cell with a compound of the formula: 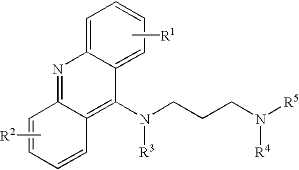
where:
R¹ and R² are independently selected from hydrogen, halogen, hydroxy, optionally substituted alkyl, optionally substituted alkyloxy, —NRR′ (where R is hydrogen or alkyl and R′ is hydrogen, alkyl, or aryl), and optionally substituted aryl; and
R₃, R₄, and R₅ are independently selected from hydrogen, optionally substituted alkyl, optionally substituted aryl, optionally substituted alkylcarbonyl, and optionally substituted arylcarbonyl,
as a single stereoisomer or mixture of stereoisomers,
or a pharmaceutically acceptable salt thereof,
in an amount sufficient to stimulate Fas-mediated apoptosis.

11. A method for obtaining and/or developing a compound that has at least one desired function selected from the group of stimulating the Fas receptor and stimulating Fas-mediated apoptosis, the process comprising administering a standard compound of the formula: 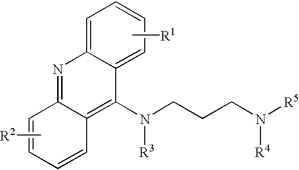
where:
R¹ and R² are independently selected from hydrogen, halogen, hydroxy, optionally substituted alkyl, optionally substituted alkyloxy, —NRR′ (where R is hydrogen or alkyl and R′ is hydrogen, alkyl, or aryl), and optionally substituted aryl; and
R₃, R₄, and R₅ are independently selected from hydrogen, optionally substituted alkyl, optionally substituted aryl, optionally substituted alkylcarbonyl, and optionally substituted arylcarbonyl,
as a single stereoisomer or mixture of stereoisomers,
or a pharmaceutically acceptable salt thereof, to an assay of Fas binding or of Fas-mediated apoptosis and noting a first result, administering a test compound to the assay and noting a second result, and comparing the first and second results, whereby a test compound producing results similar to or better than the results obtained with the standard compound has the desired function.
